(19) United States
(12) Patent Application Publication
(10) Pub. No.: US 2019/0162738 A1
(43) Pub. Date: May 30, 2019

(54) SALIVARY ABETA42 LEVELS AS PROGNOSTIC INDICATORS FOR ALZHEIMER'S DISEASE

(71) Applicant: Aurin Biotech Inc., Vancouver (CA)

(72) Inventors: Patrick L. MCGEER, Vancouver (CA); Moonhee LEE, Vancouver (CA); Jian-Ping GUO, Vancouver (CA); Krista KENNEDY, Vancouver (CA); Edith G. MCGEER, Vancouver (CA)

(21) Appl. No.: 16/321,396

(22) PCT Filed: Jul. 24, 2017
(86) PCT No.: PCT/CA2017/050883

§ 371 Date: Jan. 28, 2019

Related U.S. Application Data
(60) Provisional application No. 62/367,912, filed on Jul. 28, 2016.

(51) Int. Cl. G01N 33/68; G01N 33/577; A61P 25/28; A61K 45/00

(52) U.S. Cl. CPC G01N 33/6896; G01N 33/577; G01N 2800/50; A61K 45/00; G01N 2800/2821; A61P 25/28

(57) ABSTRACT

Methods and kits for accurate determination of salivary levels of Abeta42 are provided. A method of analysing a saliva sample includes the steps of obtaining the saliva sample from a subject; stabilizing the saliva sample; measuring the level of Abeta42 present in the stabilized saliva sample by contacting the stabilized saliva sample with an antibody capable of binding to Abeta42; comparing the determined level of the Abeta42 present in the stabilized saliva with that of a control level of Abeta42 derived from a saliva sample of an unaffected control group sample; and displaying the comparison of the determined level and the control level, wherein the determined level relative being greater than the control level is indicative of Alzheimer's disease in the subject or the subject being at risk of developing Alzheimer's disease.

### TECHNICAL FIELD

This invention relates to methods and kits for diagnosing and predicting the risk of Alzheimer's disease. More particularly, the present invention relates to the use of beta amyloid protein in saliva in diagnosis and treatment of Alzheimer's disease.

### BACKGROUND

It would be difficult to overstate the urgency of finding solutions to the problem of Alzheimer's disease (AD). Alzheimer Disease International, in their 2010 world Alzheimer Report, estimated that there were 35 million people suffering from AD, at an annual cost of $604 billion. The 2013 United States Alzheimer's Association Report estimated that there were 5.2 million cases in that country alone, with a new case being identified every 68 seconds. The annual cost in the US at that time was estimated to be $203 billion, not including the unpaid care costs of patients, which were estimated to be a further $216 billion per year (Alzheimer's Association, 2013).

AD is characterized by brain deposits of beta amyloid protein (Abeta) terminated at position 42. Abeta42 is a peptide fraction of amyloid precursor protein (APP). This fraction is relatively insoluble, and, if allowed to accumulate in brain, produces extracellular deposits in the form of senile plaques. These plaques stimulate an inflammatory response. The inflammatory response, in turn, fully activates the complement system. This results in formation of the membrane attack complex (MAC) which directly damages residual brain neurons. A progressive loss of these brain neurons occurs, which eventually results in the cognitive deficits which define clinical AD.

This sequence of events can be avoided by early intervention with anti-inflammatory agents. Traditional non-steroidal anti-inflammatory drugs (NSAIDs) are one such class of agents. Complement inhibitors are another emerging treatment class. Multiple epidemiological studies have been carried out which indicate that those consuming NSAIDs prior to the expected time of AD onset, have a substantially reduced risk of developing the disease (McGeer et al. 1996). The longer the time interval between consumption of NSAIDs and the expected onset of AD, the greater the risk reduction. It can be as much as 5-6 fold (McGeer et al. 1990). Furthermore, traditional NSAIDs were shown to have an ameliorating effect on the pathology and behavior in 13 of 14 studies using transgenic mouse models of AD. Ibuprofen was the NSAID of choice in ten of these studies (Kukar et al. 2005; McGeer and McGeer 2013).

Recent advances using biomarkers for AD have provided a plausible explanation for these results. It is now known that biomarker levels change more than a decade before the onset of AD. Bateman et al. (2012), in a landmark study of 128 cases where genetic mutations made AD development inevitable, found that concentrations of Abeta42 in the CSF declined 25 years before the expected clinical onset. Abeta deposits in the brain, as revealed by Pittsburgh compound B PET studies, were detected 15 years before expected clinical onset. Villemagne et al. (2013), estimated that it took 19.2 years of linear accumulation of Abeta deposits, 4.2 years of hippocampal atrophy, and 3.3 years of memory impairment to reach AD clinical diagnostic levels.

The basis of these predictions is that humans with certain genetic mutations in presenilin one (PS1) and presenilin two (PS2) inevitably develop AD. They result in expression of abnormally high levels of Abeta42. Transgenic mouse models that express these mutations have an overproduction of Abeta42 in brain. These are detectable as early as 2-4 months of age (Citron et al. 1997).

The opposite is observed with a mutation in the APP gene (A673T) which protects against AD. The substitution is adjacent to the beta secretase cleavage site. It results in a 40% reduction in the formation of amyloidogenic peptides in vitro (Jonsson et al. 2012).

The present inventors have determined that the window of opportunity between biomarker prediction of AD and clinical onset can be exploited to prevent AD. Intervention with non-steroidal anti-inflammatory drugs (NSAIDs), complement inhibitors, or other anti-inflammatory agents in the decade or more of preclinical development should successfully prevent AD onset. To take advantage of this opportunity, simple, inexpensive, non-invasive diagnostic methods and tests are needed that can be widely utilized by the general population.

### SUMMARY

The invention describes methods and kits for accurate determination of the level of Abeta42 in a saliva sample in a subject. The present inventors have determined that Abeta42 is produced by essentially all peripheral organs of the body and is a fundamental molecule which is continuously being generated. The present inventors have determined that typical non-AD individuals secrete low levels of Abeta42 in their saliva, regardless of sex or age, while individuals suffering from AD, and those at elevated risk of developing AD, secrete substantially higher levels of Abeta42 than these typical non-AD cases. High salivary levels of Abeta42 can therefore be used to diagnose AD and to predict the risk of future onset.

The present invention provides methods and kits for stabilizing the Abeta42 present in saliva, capturing it quantitatively, and then accurately measuring its level. The determined level of salivary Abeta42 can then be used to diagnose Alzheimer's disease (AD) in the subject. Additionally, the determined level of salivary Abeta42 can be used to evaluate risk for developing AD in the subject.

In one aspect, the method of analysing a saliva sample includes obtaining the saliva sample from a subject; stabilizing the saliva sample; measuring the level of Abeta42 present in the stabilized saliva sample by contacting the stabilized saliva sample with an antibody capable of binding to Abeta42; comparing the determined level of the Abeta42 present in the stabilized saliva with that of a control level of Abeta42 derived from a saliva sample of an unaffected control group sample; and displaying the comparison of the determined level and the control level, wherein the determined level relative being greater than the control level is indicative of Alzheimer's disease in the subject or the subject being at risk of developing Alzheimer's disease.

The present invention also provides a method of treating a subject whose salivary Abeta42 level is greater than the control level of Abeta42 derived from a saliva sample of an unaffected control group sample. The method involves administering an effective amount of NSAIDs, complement inhibitors, or other anti-inflammatory agents to reduce the risk of developing AD.

### BRIEF DESCRIPTION OF DRAWINGS

The accompanying drawings illustrate non-limiting example embodiments of the invention.

FIG. 1A is a photo showing the result of an agarose gel electrophoresis run detecting expression of amyloid precursor protein (APP) mRNAs in human organs by RT-PCR.

FIG. 1B is a photo showing the result of an agarose gel electrophoresis run detecting expression of amyloid precursor protein (APP) mRNAs in mouse organs by RT-PCR.

FIG. 2 is a standard curve plotting absorbance at 450 nm against Abeta42 concentrations in ELISA assays using a rabbit polyclonal Abeta42 antibody.

FIGS. 3A to 3D are dot blots using mouse monoclonal Abeta42. FIG. 3A is PBS buffer only. FIGS. 3B and 3C are saliva samples from normal, non-AD individuals. FIG. 3D is a saliva sample from an AD individual.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

The invention relates to non-invasive methods and related kits for diagnosing Alzheimer's disease (AD) and predicting the risk of future onset of AD. The methods and related kits are based on measuring salivary levels of beta amyloid protein terminating at position 42 (Abeta42). While it had been previously widely assumed that Abeta42 is only produced in brain (and that its appearance in blood and urine is merely the result of brain clearance), the present inventors have determined that this assumption is wrong and that many organs of the body continuously produce Abeta42, and that Abeta42 can be found in saliva. To be useful as a predictive as well as a diagnostic test for AD, the methods and kits of the present invention must be capable of measuring essentially all of the Abeta42 in a salivary sample.

According to one embodiment of the present invention, a method for diagnosing Alzheimer's disease comprises: obtaining a saliva sample from a human patient; stabilizing the saliva sample; measuring the level of Abeta42 present in the stabilized saliva sample by contacting the stabilized saliva sample with an antibody capable of binding to Abeta42; comparing the measured level with a predetermined level; and displaying a comparison of the measured level and the predetermined level.

In some embodiments, the stabilization step involves adding an anti-aggregation agent to the saliva sample to prevent Abeta42 aggregation. In an example embodiment, the anti-aggregation agent is thioflavin S. In other embodiments the anti-aggregation agent may be any suitable agent capable of preventing amyloid aggregation such as scyllo-inositol, tramiprosate (3-amino-1-propanesulfonic acid, homotaurine), curcumin, melatonin, resveratrol, and the like.

The stabilization step may, additionally or alternatively, involve adding an antimicrobial agent to prevent microbial growth in the saliva sample. In an example embodiment, the antimicrobial agent is sodium azide. In other embodiments the antimicrobial agent may be any suitable agent capable of preventing microbial growth including antibacterial and antifungal antibiotics.

The measuring step is designed to treat the stabilized saliva sample with an Abeta42 antibody in such a way as to bind essentially all of the Abeta42 present in the sample. The detection step may involve an immunoassay, such as an ELISA test based on an antigen-antibody reaction using Abeta42 as the antigen to be measured.

For example, the capture antibody may be a polyclonal antibody specific for Abeta42. The bound Abeta42 is then detected by a second Abeta42 antibody which does not cross react with the first Abeta42 antibody. In some embodiments the second Abeta42 antibody may be a monoclonal antibody, such as a mouse monoclonal antibody. Biotin may then be bound to the monoclonal antibody or to a third IgG antibody to which biotin is bound. The biotin levels are detected by first treating with a streptavidin solution linked with horse radish peroxidase (HRP) followed by treatment with a tetramethylbenzidine solution to detect the HRP by reading the resulting color in a spectrophotometer.

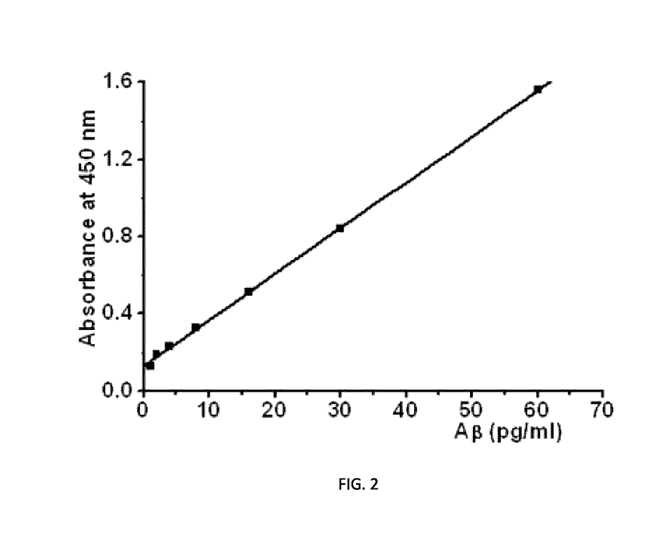

An ELISA standard concentration graph of target Abeta42 may be obtained, for example, by absorbance detection, fluorescence detection, luminescence detection, or electrochemical detection. FIG. 2 shows a standard concentration graph for Abeta42 obtained by absorbance at 450 nm. Alternatively, the immunoassay may be performed by a method capable of directly or indirectly detecting Abeta42. Examples of such alternative methods include MS (Mass Spectrometry), MS/MS, and liquid chromatography.

The comparison step may involve comparing the measured level of Abeta42 obtained from the detection step against a predetermined level of Abeta42. The predetermined level of Abeta42 may be obtained from concurrent or previous saliva samples from normal individuals without AD and without any genetic predisposition for AD.

The displaying step involves displaying values, diagrams, illustrations, and the like. This step is preferably a step that can assist in assessing the difference between the measured level and the predetermined level. For example, the difference may be displayed by the ratio of the measured level to the predetermined level. A diagnosis of AD, or a prediction of risk of future onset of AD, may be made, for example, if this ratio exceeds 1.5, or 2.0, or 2.5.

In another embodiment of the invention, a qualitative screening test is provided. For example, the test may comprise a dot blot as described in Example 5 below. Such tests can be used by individuals to determine whether quantitative determination of salivary Abeta42 levels is warranted.

In another embodiment of the invention, a method of treating a subject whose salivary Abeta42 level is greater than the control level of Abeta42 derived from a saliva sample of an unaffected control group sample is provided. When the ratio of the measured salivary Abeta42 level to the predetermined level exceeds 1.5, 2.0, or 2.5, an effective amount of NSAIDs, complement inhibitors, or other anti-inflammatory agents is administered to the subject to prevent AD onset. Multiple epidemiological studies have suggested that those consuming anti-inflammatory agents, such as NSAIDs, prior to the expected time of AD onset, have a substantially reduced risk of developing the disease (McGeer et al. 1996).

The invention can be further understood by reference to the following examples, which are provided by way of illustration and are not meant to be limiting.

### Example 1—Detecting APP mRNA and Abeta42 Peptide in Organs

The present inventors first investigated whether or not mRNA for APP was present in organs other than the brain.

Frozen samples of human organs and mouse brains were utilized as the starting material. The samples were homogenized by a sonic dismembrator (Fisher Scientific, Ottawa, ON). They were then treated with 100 microliters of TRIzol (GIBCO-BRL, Gathersburg, Md.). After 1 h, the lysates were centrifuged at 10,000 rpm for 10 min. The supernatants were transferred to new tubes. The purity and amount of the RNA was then measured spectrophotometrically.

A total RNA aliquot (20 pg) was used to synthesize the first strand complementary DNA (cDNA) using Moloney murine leukemia virus (M-MLV) reverse transcriptase (GIBCO-BRL). The cDNA product was then amplified by PCR using a GeneAmp thermal cycler (Applied Biosystems, Foster City, Calif.).

Specific primers for human βAPP and GAPDH are βAPP: Forward 5′-CGGAATTCCCTTGGTGTTCTTTGCAGAAG-3′ and Reverse 5′-CGGAATTCCGTTCTGCATCTGCTCAAAG-3′ (248 bp) (Miklossy et al., 2010), and GAPDH: Forward 5″-CCATGTTCGTCATGGGTGTGAACCA-3″ and Reverse 5′-GCCAGTAGAGGCAGGGATGATGTTC-3″ (251 bp) (Lee et al., 2010). Specific primers for mouse βAPP and GAPDH are βAPP: Forward 5′CCACCACAACCACCACTGAG-3′ and Reverse 5′CTGGATACACCCGGGGACGA-3′ (331 bp for APP770, 274 bp for APP751 and 107 bp for APP695) (Jeong et al., 1997), and b-actin: Forward 5′-GGCTGCTTTTGTAGGCTTCAGTGG-3″ and Reverse 5′-TCTAGACTTCGAGCAGGAGATGGCC-3″ (739 bp) (Matsumura et al., 2004). PCR conditions for human organs were an initial denaturation step at 95° C. for 6 min followed by a 30-cycle amplification program consisting of denaturation at 95° C. for 45 sec, annealing at 55-60° C. for 1 min and extension at 72° C. for 1 min. A final extension was carried out at 72° C. for 10 min. For mouse organs, the PCR conditions were an initial denaturation at 95° C. for 1 min followed by a 30-cycle amplification program, consisting of denaturation at 95° C. for 45 sec, annealing at 55° C. for 1 min and extension at 72° C. for 2 min. A final extension was carried out at 72° C. for 10 min. The amplified PCR products were identified using 1.5% agarose gels containing ethidium bromide (final concentration 0.5 pg/ml) and visualized under ultraviolet light.

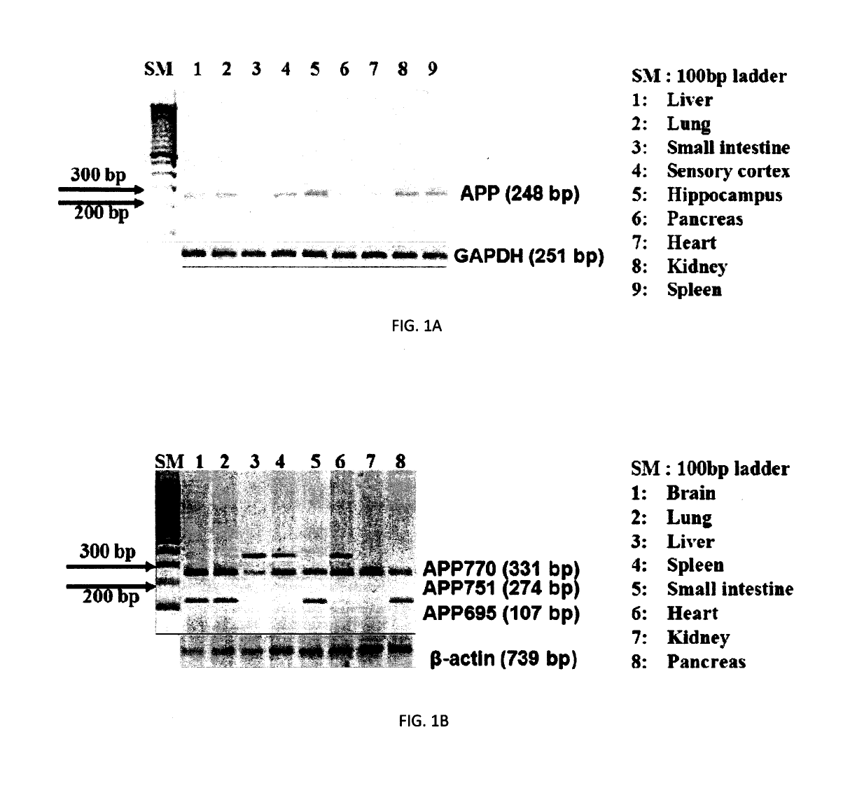

As shown in FIGS. 1A and 1B, m RNA for APP, which can be translated into Abeta42, was expressed in all human and mouse organs tested. Expression of GAPDH and β-actin loading controls shown in the lower panels were used as standards for human and mouse, respectively.

The present inventors next demonstrated that this mRNA was translated into protein which resulted in Abeta42 being detected in all body organs tested, as shown in Table 1, below. Frozen tissues from human liver, lung, small intestine, sensory cortex, hippocampus, pancreas, heart, kidney, spleen as well as 3 mouse brains were thawed and dissected into small pieces. The dissected pieces were added to chilled tubes with a tenfold excess of 0.01 M phosphate buffered saline (PBS, PH 7.4). The mixture was sonicated ten times at 20% amplitude for 25 seconds (Branson Digital Sonifier, USA). The mixture was then centrifuged at 10,000 g for 30 minutes at 4° C. The samples were analyzed using immunoassays according to the present invention (and described further below with respect to the saliva samples).

Thus, while it had been previously widely assumed that Abeta42 is only produced in brain (and that its appearance in blood and urine is merely the result of brain clearance), the present inventors have therefore determined that this assumption is wrong and that many organs of the body continuously produce Abeta42.

TABLE 1
Amount of Abeta42 in various human organs (upper panel) and
mouse brains (lower panel) using the invention methodology
compared with a commercial kit

| | Abeta42 levels | Abeta42 levels |
|---|---|---|
| | (our invention) | (Invitrogen kit) |
| | picograms/mg | picograms/mg |
| Human tissue | | |
| Lung | 83.68 | 17.28 |
| heart | 88.59 | 18.29 |
| kidney | 122.65 | 29.22 |
| hippocampus | 102.45 | 18.28 |
| sensory cortex | 97.83 | 18.24 |
| liver | 90.56 | 17.59 |
| spleen | 134.38 | 29.49 |
| small intestine | 80.58 | 18.02 |
| pancreas | 128.22 | 40.1 |
| Mouse brain | | |
| Mouse 1 | 4.35 | 0.37 |
| Mouse 2 | 3.84 | 0.51 |
| Mouse 3 | 3.98 | 0.93 |

Table 1 also compares results obtained using the method of the invention compared with those obtained using a commercial ELISA kit from Invitrogen (Frederick, Md.). The manufacturer's instructions were closely followed. As can be seen from Table 1, the commercial ELISA kit picks up only about 20% of the Abeta42 present in tissues as determined by the method of the invention.

### Example 2—Quantitative Detection of Abeta42 Peptide in Saliva

To determine Abeta42 content in saliva, 100 μL of a rabbit polyclonal anti-Abeta42 antibody diluted 1/1000 in PBS was added to 96 well plates and left overnight at 4° C. to allow the antibody to bind to the plates. In this case, the primary or capture antibody was a rabbit polyclonal Abeta42 antibody obtained from Novus Biochemicals (NBP2-44113, Littleton Colo.). The supernatant was then discarded. Next, to block non-specific binding to the capture antibody, 400 μL of 5% bovine serum albumin (BSA, Sigma, St. Louis, Mo.) was added to the plates, followed by incubation at 37° C. for 1 h. The BSA solution was discarded and the wells were washed twice with PBS.

Saliva at various concentrations (0-100 μL) was added to the plates and incubated at 37° C. for 4 h. The supernatants were then discarded. The plates were washed twice with PBS. They were next incubated with a mouse monoclonal antibody to Abeta42. In this case the antibody was from Anaspec (Fremont, Calif.). It was added as 100 μL of a 1/200 dilution of 5% BSA in PBS. Incubation was carried out at 37° C. for 2 h. The plates were washed twice with PBS. They were next incubated at 37° C. for 1 h with 100 μL of a biotin-linked rabbit anti-mouse IgG antibody (DAKO, Mississauga, ON, Canada) diluted 1/200 in PBS containing 5% BSA. The supernatants were discarded.

To detect the bound biotin, a streptavidin solution (ABC, 1/1000 dilution, Vector Laboratories) was added and incubation carried out at 37° C. for 30 minutes. The plates were washed twice with PBS, a tetramethybenzidine (TMB) solution (100 μL, Invitrogen, Carlsbad, Calif.) was added and incubation carried out at 37° C. for a further 30 min. The reaction was terminated and optical density measured at 450 nm.

The present inventors then demonstrated the effects of stabilizing the Abeta42 present in saliva. Two mL of saliva were collected per container. For the control samples, no stabilizers were added. For the test samples, thioflavin S (0.5 mg, Sigma St. Louis Mo.) was immediately added to prevent Abeta42 aggregation, and sodium azide (0.5 mg, Fischer Scientific, Suwanee Ga.) was also added to prevent bacterial growth. The containers were left for 7 days at room temperature while aliquots were taken periodically for Abeta42 and protein measurement.

As the results in Table 2 show, Abeta42 without stabilizers deteriorates rapidly. Within 3 days at ambient temperature, the values had deteriorated to well below 1% of those originally present. However with thioflavin S and sodium azide added, there was complete stability after 7 days at ambient temperature, and for an undetermined time thereafter. This stabilization makes it practical to store and/or transport saliva. For example, logistical issues, sample processing capacity issues and the like may dictate that samples are not analyzed for days. The stabilization step is thus critical in embodiments where the analysis step occurs at least one day, or at least two days, or at least three days, or at least five days, or at least one week, after sample collection and storage at ambient temperature.

A suitable reference standard was also identified to compensate for any dilution of the saliva at the time of collection, as shown in the last three columns of Table 2. Total protein in the sample proved to be such a standard. A standard method of protein measurement was followed. Concentrations were determined using an assay kit provided by Thermo Scientific Inc. (Rockford, Ill.). The kit is based on reactions with bicinchoninic acid. Bovine serum albumin is the reference standard. Saliva samples or PBS as controls (10 μL) were added to microwell plates. Incubation was carried out at 37° C. for 30 minutes. The optical density (OD) was then read at 562 nm.

TABLE 2
Abeta42 and protein levels with and without thioflavin
S and sodium azide as preservatives.

| | | Amount of Abeta42 in pg/ml | | | Amount of protein in mg/ml | | |
|---|---|---|---|---|---|---|---|
| time | | Case 1 | Case 2 | Case 3 | Case 1 | Case 2 | Case 3 |
| Day 0 | With | 23.94 | 24.56 | 25.89 | 2.31 | 2.095 | 2.36 |
| | preservatives | | | | | | |
| | Without | 23.69 | 24.51 | 25.57 | 2.38 | 2.08 | 2.36 |
| | preservatives | | | | | | |
| Day 1 | With | 23.33 | 23.95 | 24.88 | 2.42 | 2.16 | 2.27 |
| | preservatives | | | | | | |
| | Without | 18.53 | 16.52 | 16.94 | 2.73 | 2.41 | 2.71 |
| | preservatives | | | | | | |
| Day 2 | With | 22.93 | 23.12 | 24.25 | 2.31 | 2.08 | 2.23 |
| | preservatives | | | | | | |
| | Without | 7.93 | 7.59 | 6.92 | 2.96 | 2.79 | 2.93 |
| | preservatives | | | | | | |
| Day 3 | With | 22.94 | 23.46 | 23.86 | 2.38 | 2.14 | 2.42 |
| | preservatives | | | | | | |
| | Without | 2.94 | 2.18 | 2.09 | 3.04 | 3.60 | 3.34 |
| | preservatives | | | | | | |
| Day 5 | With | 22.12 | 23.45 | 23.58 | 2.36 | 2.28 | 2.22 |
| | preservatives | | | | | | |
| | Without | 0.12 | 0.15 | 0.15 | 3.44 | 3.79 | 3.63 |
| | preservatives | | | | | | |
| Day 7 | With | 21.55 | 22.84 | 22.69 | 2.32 | 2.21 | 2.24 |
| | preservatives | | | | | | |
| | Without | 0 | 0 | 0 | 3.49 | 3.91 | 3.58 |
| | preservatives | | | | | | |

### Example 3—Detecting and Predicting AD

Table 3 illustrates the diagnosis and predictability of AD by measuring salivary Abeta42 levels in a series of individuals. The Table shows no overlap of control and AD values indicating that the invention can reliably diagnose and predict for each person in which a measurement is carried out.

TABLE 3
Abeta42 levels in both normal and AD cases measured by our
ELISA-type assays.

| Case | Age | Gender | Diagnosis | Abeta42 (pg/ml) |
|---|---|---|---|---|
| 1 | 46 | Male | Normal | 25.32 |
| 2 | 53 | Male | Normal | 24.44 |
| 3 | 89 | Male | Normal | 22.35 |
| 4 | 16 | Male | Normal | 21.47 |
| 5 | 19 | Male | Normal | 20.27 |
| 6 | 19 | Female | Normal | 19.73 |
| 7 | 22 | Male | Normal | 22.27 |
| 8 | 24 | Male | Normal | 21.53 |
| 9 | 39 | Male | Normal | 22.22 |
| 10 | 42 | Male | Normal | 20.58 |
| 11 | 47 | Female | Normal | 20.15 |
| 12 | 54 | Male | Normal | 20.05 |
| 13 | 54 | Female | Normal | 20.56 |
| 14 | 55 | Female | Normal | 29.66 |
| 15 | 54 | Male | PD | 22.32 |
| 16 | 57 | Female | Normal | 19.61 |
| 17 | 57 | Female | Normal | 23.07 |
| 18 | 59 | Male | Normal | 21.55 |
| 19 | 60 | Male | Normal | 22.02 |
| 20 | 62 | Male | Normal | 21.37 |
| 21 | 78 | Male | Normal | 21.44 |
| 22 | 83 | Female | Normal | 20.56 |
| 23 | 86 | Male | Normal | 21.37 |
| 24 | 88 | Male | Normal | 22.46 |
| 25 | 92 | Female | Normal | 20.89 |
| 26 | 52 | Female | Pre-AD | 59.57 |
| 27 | 60 | Female | Pre-AD | 47.96 |
| 28 | 57 | Male | AD | 41.58 |
| 29 | 72 | Female | AD | 41.69 |
| 30 | 77 | Female | AD | 84.97 |
| 31 | 78 | Male | AD | 48.89 |
| 32 | 84 | Female | AD | 64.93 |
| 33 | 86 | Female | AD | 75.20 |

AD: Alzheimer's disease and PD: Parkinson’s disease. 33 cases were tested. Controls (including 1 PD case), 25. AD, 6 cases.
Mean ± SEM: controls, 23.34 ± 1.50; AD, 57.89 ± 6.53. One-way ANOVA was carried out to test the significance of the difference between AD and control cases: for data P < 0.001 between controls and AD.

The method described in Example 2 was utilized to determine the Abeta42 levels in a series of 33 individuals as shown in Table 3. As the Table illustrates, non-AD cases (controls) expressed levels that were remarkably constant over ages from 16 to 92 with no difference between males and females. The 25 non-AD cases, including one with Parkinson disease, showed a mean of 23.34±1.50 picograms per ml. The 6 AD cases showed a mean of 57.89±6.53 picograms per ml. One-way ANOVA was carried out to test the significance of differences between the control and AD cases (p<0.001). There was no overlap between AD and non-AD cases except for cases 26 and 27. They had values in the AD range and were designated as Pre-AD. Both cases had mothers affected by AD, so they were known to be at a higher than normal risk to develop AD. The salivary Abeta42 levels establish that in their cases the risk is definite rather than possible. Overall, these data demonstrate that the invention can diagnose AD as well as predict the risk of future onset.

### Example 4—Qualitative Detection of AD

In order to provide a quick and qualitative way to detect AD, the present inventors assessed the feasibility of a dot blot test. Small strips of nitrocellulose membranes were saturated with the rabbit polyclonal Abeta42 antibody (Novus Biologicals) at 1/1000 dilution. Small dots of saliva were then applied. After drying, the membrane was treated with a blocking solution (5% BSA in PBS) for 1 h at room temperature. Mouse monoclonal Abeta42 antibody (Anaspec, 1/200 dilution) was then applied to the dot areas followed by application of rabbit polyclonal anti mouse IgG antibody 6 E10 (1/200). After washing, the plate was incubated with the biotin-linked rabbit anti-mouse IgG antibody (1/200 in 5% (DAKO, Mississauga, ON, Canada) in 3% BSA, at 37° C. for 1 h. HRP-linked streptavidin solution (ABC solution, 1/1000 dilution, Vector Laboratories) was added and incubation carried out at 37° C. for 30 min. The color reaction was performed with an enhanced chemiluminescence system (Luminata™ Crecendo Western HRP Substrates, Millipore, Billerica, Mass.) for 5 min.

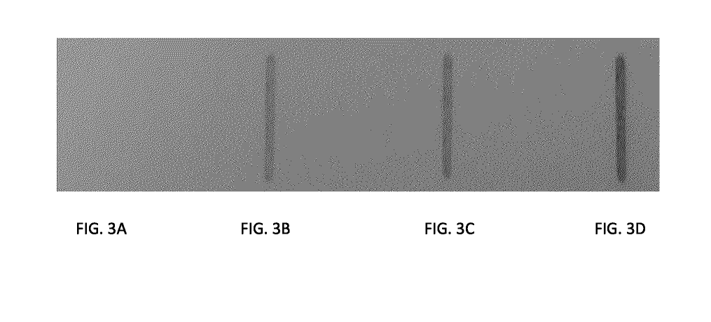

FIGS. 3A to 3D show results of the dot blots of salivary Abeta42 in typical normal and a typical AD cases. There was approximately 2.7 times more Abeta42 detected in the AD case than in the two normal cases.

TABLE 4
Abeta42 recovery study data from three normal and three AD cases.

| | Abeta42 amount (pg/ml) | | | | | |
|---|---|---|---|---|---|---|
| Cases | CON 1 | CON 2 | CON 3 | AD 1 | AD 2 | AD 3 |
| Aβ (25 pg/ml) | 25.33 | 24.89 | 25.12 | 25.33 | 24.94 | 25.04 |
| Saliva | 24.56 | 24.17 | 23.22 | 85.01 | 65.07 | 75.17 |
| Saliva + Aβ | 49.35 | 49.02 | 48.85 | 109.72 | 91.46 | 100.04 |
| (25 pg/ml) | | | | | | |

CON: normal and AD: Alzheimer disease. Note that mean recovery and SEM of Aβ (25 pg/ml) is 25.11 ± 0.08 pg/ml, n = 6.

Where a component is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

This application is intended to cover any variations, uses, or adaptations of the invention using its general principles. Further, this application is intended to cover such departures from the present disclosure as come within known or customary practice in the art to which this invention pertains and which fall within the limits of the appended claims. Accordingly, the scope of the claims should not be limited by the preferred embodiments set forth in the description, but should be given the broadest interpretation consistent with the description as a whole.

### REFERENCES

Bateman R J, Xiong C, Benziger T L S et al. (2012). Clinical and biomarker changes in dominantly inherited Alzheimer's disease. New Engl J Med 367(9):793-804.
Bermajo-Parejo F, Antequera D, Vargas J A et al. (2010). Saliva levels of Abeta1-42 as potential biomarker of Alzheimer's disease: a pilot study. BMC Neurol 10:108.
Citron M, Westaway D, Xia W et al. (1977). Mutant presenilins of Alzheimer disease increase production of 42-residue amyloid b-protein in both transfected cells and transgenic mice. Nature Medicine 3: 67-72.
Jeong S J, Kim K, Suh Y (1997). Age-related changes in expression of Alzheimer beta APP in the brains of senescence accelerated mouse SAM-P/10. Neuroreport 8: 1733-1737.
Jonsson T, Atwal J K, Steinberg S, et al. (2012). A mutation in APP protects against Alzheimer's disease and age-related cognitive decline. Nature 488 (7409): 96-99.
Kukar T, Murphy M P, Erikson J L et al. (2005) Diverse compounds mimic Alzheimer disease-causing mutations by augmenting Aβ 42 production. Nature Med 11:545-550.
Lee M, Cho T, Jantaratnotai N, Wang Y T, McGeer E, McGeer P L (2010). Depletion of GSH in glial cells induces neurotoxicity: relevance to aging and degenerative neurological diseases. FASEB J. 24(7):2533-2545.
Matsumura R, Takeuchi S, Takahashi S (2004). Effect of estrogen on melanocortin-3 receptor mRNA expression in mouse pituitary glands in vivo and in vitro. Neuroendocrinology 80(3): 143-151.
McGeer P L, McGeer E G. (2013). The amyloid cascade-inflammatory hypothesis of Alzheimer disease. Acta Neuropath 126(4): 479-497.
McGeer P L, McGeer E G, Rogers J, Sibley J. (1990). Anti-inflammatory drugs and Alzheimer disease. Lancet 335:107.
McGeer P L, Schulzer M, McGeer E G. (1996). Arthritis and anti-inflammatory agents as negative risk factors for Alzheimer disease: A review of seventeen epidemiological studies. Neurology 47:425-432.
Miklossy J, Qing H, Radenovic A, et al. (2010) Beta amyloid and hyperphosphorylated tau deposits in the pancreas in type 2 diabetes. Neurobiol Aging. 31(9):1503-1515.
Alzheimer's Association (2013). Alzheimer's disease facts and figures. Alzheimer's Dem 9:208-245.
Villemagne V L, Burnham S, Bourgeat P, et al. (2013). Amyloid β deposition, neurodegeneration, and cognitive decline in sporadic Alzheimer's disease: a prospective cohort study. Lancet Neurol. 12(4): 357-367.

1. A method of analyzing a saliva sample to diagnose Alzheimer's disease in a subject, the method comprising:
obtaining the saliva sample from the subject;
stabilizing the saliva sample;
measuring the level of Abeta42 present in the stabilized saliva sample by contacting the stabilized saliva sample with an antibody capable of binding to Abeta42;
comparing the determined level of the Abeta42 present in the stabilized saliva with that of a control level of Abeta42 derived from a saliva sample of an unaffected control group sample; and
displaying the comparison of the determined level and the control level, wherein the determined level being greater than the control level is indicative of Alzheimer's disease in the subject.

2. A method according to claim 1 wherein the determined level relative being at least 1.5 times greater than the control level is indicative of Alzheimer's disease in the subject.

3. A method according to claim 2 wherein the determined level relative being at least twice the control level is indicative of Alzheimer's disease in the subject.

4. A method according to claim 1 wherein the stabilizing step comprises adding an anti-aggregation agent.

5. A method according to claim 4 wherein the anti-aggregation agent is thioflavin S.

6. A method according to claim 1 wherein the stabilizing step comprises adding an antimicrobial agent.

7. A method according to claim 6 wherein the antimicrobial agent is sodium azide.

8. A method according to claim 1 wherein the antibody comprises a non-human polyclonal antibody.

9. A method according to claim 1 wherein the measuring step, after contacting the stabilized saliva sample with the antibody capable of binding to Abeta42, comprises contacting the stabilized saliva sample with a second antibody capable of binding to Abeta42, wherein the second antibody is a non-cross reacting antibody.

10. A method according to claim 9 wherein the non-cross reacting antibody comprises a non-human monoclonal antibody.

11. A method for evaluating risk for developing Alzheimer's disease in a subject, the method comprising:
obtaining a saliva sample from the subject;
stabilizing the saliva sample;
measuring the level of Abeta42 present in the stabilized saliva sample by contacting the stabilized saliva sample with a non-human antibody capable of binding to Abeta42;
comparing the determined level of the Abeta42 with that of a control level of Abeta42 derived from a saliva sample of an unaffected control group sample; and
displaying the comparison of the determined level and the control level, wherein the determined level being greater than the control level is indicative of the subject being at risk for developing Alzheimer's disease.

12. A method according to claim 11 wherein the determined level relative being at least 1.5 times greater than the control level is indicative of the subject being at risk for developing Alzheimer's disease.

13. A method according to claim 12 wherein the determined level relative being at least twice the control level is indicative of the subject being at risk for developing Alzheimer's disease.

14. A method according to claim 11 wherein the stabilizing step comprises adding an anti-aggregation agent.

15. A method according to claim 14 wherein the anti-aggregation agent is thioflavin S.

16. A method according to claim 11 wherein the stabilizing step comprises adding an antimicrobial agent.

17. A method according to claim 16 wherein the antimicrobial agent is sodium azide.

18. A method according to claim 11 wherein the non-human antibody comprises a polyclonal antibody.

19. A method according to claim 11 wherein the measuring step, after contacting the stabilized saliva sample with the antibody capable of binding to Abeta42, comprises contacting the stabilized saliva sample with a second non-human antibody capable of binding to Abeta42.

20. A method according to claim 19 wherein the second non-human antibody comprises a monoclonal antibody.

21. A method of measuring the level of salivary Abeta42 in a subject, the method comprising:
obtaining a saliva sample from the subject;
stabilizing the saliva sample; and
contacting the stabilized saliva sample with a non-human polyclonal antibody capable of binding to Abeta42; and
contacting the stabilized saliva sample with a non-human monoclonal antibody capable of binding to Abeta42.

22. The method according to claim 21 wherein the non-human polyclonal antibody is a rabbit polyclonal antibody.

23. The method according to claim 21, wherein the non-human monoclonal antibody is a mouse monoclonal antibody.

24. A kit for diagnosing Alzheimer's disease in a subject, the kit comprising:
a sterile plastic tube for collecting a saliva sample from the subject;
at least one binding agent, wherein the binding agent is an antibody capable of binding to salivary Abeta42;
a calibration standard; and
a control saliva sample obtained from an unaffected control group sample.

25. A kit according to claim 24 wherein the sterile plastic tube is pre-filled with an anti-aggregation agent.

26. A kit according to claim 24 wherein the anti-aggregation agent comprises thioflavin S.

27. A kit according to claim 24 wherein the sterile plastic tube is pre-filled with an antimicrobial agent.

28. A kit according to claim 26 wherein the antimicrobial agent comprises sodium azide.

29. A kit according to claim 24 wherein the binding antibody comprises a non-human polyclonal antibody.

30. A kit according to claim 29 wherein the non-human polyclonal antibody is a rabbit polyclonal antibody.

31. A kit according to claim 24 wherein the binding antibody comprising a non-human monoclonal antibody.

32. A kit according to claim 31 wherein the non-human monoclonal antibody is a mouse monoclonal antibody.

33. (canceled)

34. (canceled)

35. A method of reducing the risk of developing Alzheimer's disease in a subject, comprising:
obtaining a saliva sample from the subject;
stabilizing the saliva sample;
measuring the level of Abeta42 present in the stabilized saliva sample by contacting the stabilized saliva sample with a non-human antibody capable of binding to Abeta42;
comparing the determined level of the Abeta42 with that of a control level of Abeta42 derived from a saliva sample of an unaffected control group sample;
displaying the comparison of the determined level and the control level, wherein the determined level being greater than the control level by at least a factor of 1.5 or at least a factor of 2 is indicative of the subject being at risk for developing Alzheimer's disease; and
reducing the risk of developing Alzheimer's disease in the subject being at risk for developing Alzheimer's disease by administering to an subject anti-inflammatory agent.

36. A method according to claim 35 wherein the anti-inflammatory agent is a non-steroidal anti-inflammatory drugs (NSAID) or complement inhibitor.
